(19) United States
(12) Patent Application Publication
(10) Pub. No.: US 2025/0145603 A1
(43) Pub. Date: May 8, 2025

(54) CRYSTAL FORMS OF AN ANTI-RSV AGENT

(71) Applicant: Enanta Pharmaceuticals, Inc., Watertown, MA (US)

(72) Inventors: Joseph HELBLE, Hudson, MA (US); Scott CRAWFORD, Southborough, MA (US); Andrew HAGUE, Chelmsford, MA (US)

(21) Appl. No.: 18/938,551

(22) Filed: Nov. 6, 2024

Related U.S. Application Data
(60) Provisional application No. 63/547,586, filed on Nov. 7, 2023.

(51) Int. Cl. C07D 413/14

(52) U.S. Cl. CPC C07D 413/14

(57) ABSTRACT

The present invention features crystalline forms of Compound I having characteristic peaks in the XRPD pattern as shown in one of FIGS. 1-9 and Tables 2-5.

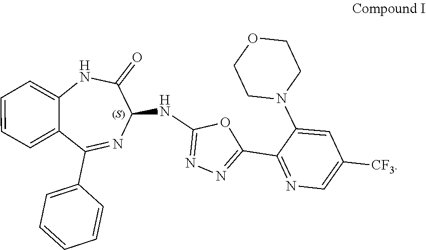

### RELATED APPLICATION

This application claims the benefit of U.S. Provisional Application No. 63/547,586, filed on Nov. 7, 2023. The entire teachings of the above application are incorporated herein by reference.

### FIELD OF THE INVENTION

The present invention relates to crystalline polymorphic forms of Compound I, pharmaceutical compositions comprising the same, and methods of using the same to prepare pharmaceutical compositions. Compound I is useful for the treatment of RSV (Respiratory Syncytial Virus).

### BACKGROUND

Respiratory Syncytial Virus (RSV) is a virus that infects the lungs and represents a serious unmet medical need in infants and children, as well as immune-compromised individuals and the elderly. RSV is the most common cause of bronchiolitis and pneumonia in children under the age of one. In one large U.S.-based study, RSV infection in children was associated with 20% of hospitalizations, 18% of emergency department visits, and 15% of pediatric office visits for acute respiratory infections in the November-April time frame. Though a prophylactic monoclonal antibody-based treatment is available for those considered at high risk for RSV infection, the study (palivizumab) found that most young children affected by RSV infection were previously healthy, and thus would not normally be considered for prophylaxis. There is currently no safe and effective treatment available for treating RSV infection. Compound I is a potent non-fusion inhibitor of both RSV-A and RSV-B.

### SUMMARY OF THE INVENTION

The present invention provides crystalline polymorphs of Compound I [Chemical name: (S)-3-((5-(3-morpholino-5-(trifluoromethyl) pyridin-2-yl)-1,3,4-oxadiazol-2-yl)amino)-5-phenyl-1,3-dihydro-2H-benzo[e][1,4]diazepin-2-one], which has the structure below.

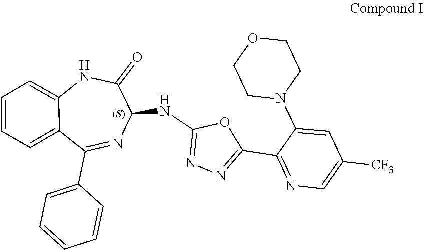

In certain embodiments, the invention provides Compound I in a polymorphic or pseudopolymorphic form as disclosed herein.

In certain embodiments, the invention provides methods of producing the polymorphs and pseudopolymorphs of Compound I disclosed herein.

In certain embodiments, the invention provides compositions comprising a polymorph or pseudopolymorph of Compound I disclosed herein. In certain embodiments, the composition is a pharmaceutical composition comprising at least one polymorph or pseudopolymorph of Compound I and a pharmaceutically acceptable carrier or excipient. In certain embodiments, the composition is substantially free of other polymorphs or pseudopolymorphs of Compound I.

In certain embodiments, the invention provides a method of treating or preventing a respiratory syncytial virus infection in a subject in need thereof. The method comprises the step of administering to the subject (a) a therapeutically effective amount of a polymorph or pseudopolymorph of Compound I, (b) a therapeutically effective amount of two or more polymorph or pseudopolymorph forms of Compound I, or (c) a therapeutically effective amount of one or more polymorphs and/or pseudopolymorphs of Compound I and an amorphous form of Compound I.

### BRIEF DESCRIPTION OF THE DRAWINGS

FIG. 1 shows the X-ray powder diffraction (XRPD) pattern of crystalline Form A of Compound I.

FIG. 2 shows the XRPD pattern of crystalline Material B of Compound I.

FIG. 3 shows the XRPD pattern of crystalline Form A+Material C of Compound I.

FIG. 4 shows the XRPD pattern of crystalline Form D of Compound I.

FIG. 5 shows the XRPD pattern of crystalline Form A+Material E of Compound I.

FIG. 6 shows the XRPD pattern of crystalline Form F of Compound I.

FIG. 7 shows the XRPD pattern of crystalline Form G of Compound I.

FIG. 8 depicts XRPD patterns of the following forms of Compound I from top to bottom: Material B; Form G; Form F; Form D; Form A+Material E; Form A+Material C; Form A.

FIG. 9 shows the XRPD pattern of Compound I Amorphous Form.

### DETAILED DESCRIPTION OF THE INVENTION

Compound I is a potent RSV N-protein inhibitor which can be prepared using the methods disclosed in WO 2017/015449 and WO 2018/152413, both of which are incorporated herein by reference in their entirety. Compound I is a single enantiomer. It has one chiral center, the 3-position of the 1,3-dihydro-2H-benzo[e][1,4]diazepin-2-one ring, which has the(S) configuration of the chiral center.

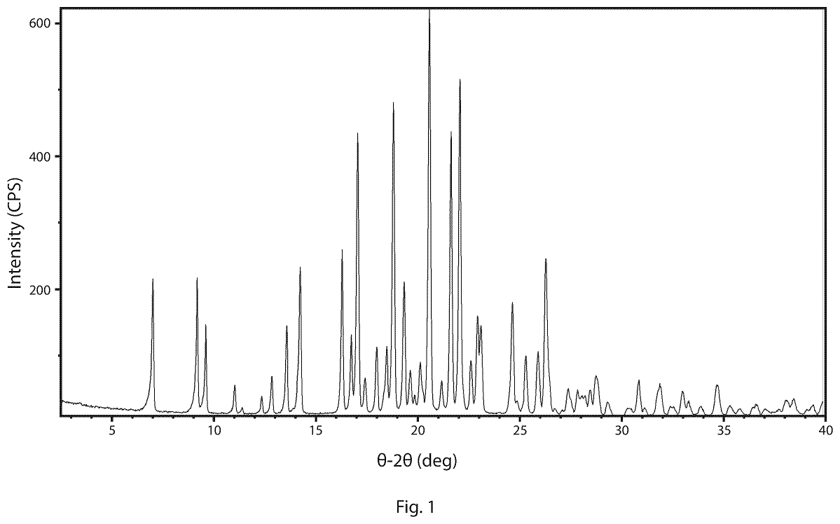

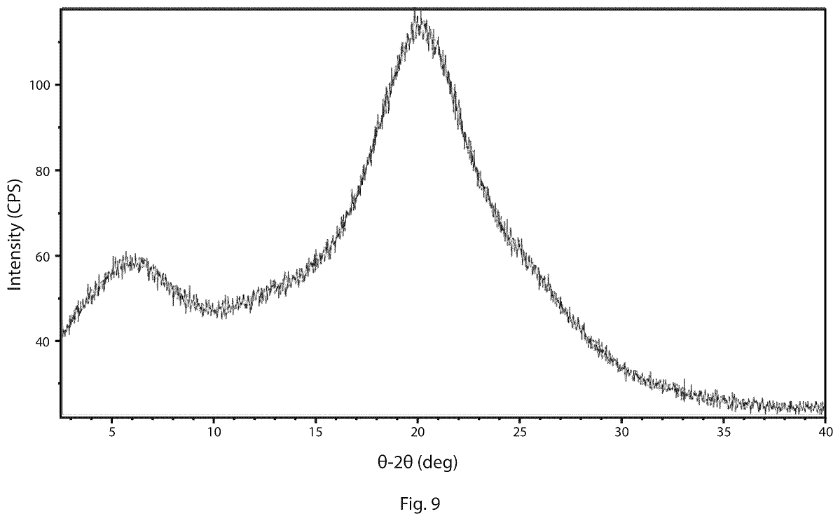

In addition to the amorphous form having the XRPD shown in FIG. 9, seven other solid forms were observed in a polymorph screen of Compound I. These solid forms are designated as Form A, Material B, Material C, Form D, Material E, Form F, and Form G, which are summarized in Table 1 below. Materials C and E were only observed as mixtures with Form A. The relative intensity of each peak in FIGS. 1-9 may change or shift under certain conditions, although the crystalline form is the same. One of the ordinary skill in the art will be able to readily determine whether a given crystalline form is the same crystalline form as described in one of FIGS. 1-9 by comparing its XRPD pattern with FIGS. 1-9, Table 1, or Tables 2-5.

TABLE 1
Summary of Observed Compound I Solid Forms/Materials

| Compound I | Observed alone or in | |
|---|---|---|
| Solid Form | mixture with Form A | Properties |
| Amorphous | Either | Appeared non-hygroscopic |
| | | Crystallizes to A |
| Form A | — | Confirmed single phase |
| | | Crystal structure known |
| | | Anhydrous/unsolvated, non-hygroscopic |
| | | Physically stable below 100% RH |
| Material B | Either, but alone only from | Potential toluene solvate |
| | slurrying A/B mixture in | Stability unknown |
| | methanol/toluene | |
| Material C | Mixture | Appears to be single phase |
| | | Potential ethanolate |
| | | Likely converts to A on drying |
| | | at ambient temperature |
| Form D | Alone, from slurrying in | Confirmed single phase |
| | methanol | Likely methanolate |
| | | Converts to F upon air drying |
| Material E | Mixture, from slurrying A in | Appears to be single phase |
| | aqueous tetrahydrofuran at | Nature of E is not fully determined, |
| | water activity of 1.19 | could be solvated, hydrated, |
| | | or mixed solvate/hydrate |
| Form F | Alone, from air drying D | Confirmed single phase |
| | | Converts to G in ethyl acetate |
| Form G | Either, from slurrying F | Confirmed single phase |
| | in ethyl acetate | Likely anhydrous/unsolvated |
| | Alone, when only F in slurry | May be epimerized |
| | Mixture, when A/F in slurry | |

The designation is tentatively associated with the term “Material” until the phase purity and chemical composition is determined through further characterization. Identification as Compound I and verification of phase uniformity are necessary before the word “Form” is used. As part of the polymorph screen, amorphous solids were also generated and used as the starting material for a crystallization study, which yielded Form A under most of the conditions examined.

Form A is a crystalline form and the preferred form of Compound I. Preliminary stability data show Form A is a stable polymorph form. Form A is an off-white to yellow solid that is not hygroscopic and has a melting point range of 227-230° C. It is highly soluble in organic solvents including, but not limited to, dichloromethane, 1,4-dioxane, tetrahydrofuran, and acetone, but has relatively poor solubility in organic solvents including, but not limited to. alcohols, ethyl acetate and acetonitrile. In water, Form A has a solubility of approximately 2 μg/mL from pH 4-8 and 13.3 μg/mL at pH 1. Form A is an anhydrous, un-solvated crystalline solid, which can be produced as a single crystalline phase that is shown to be stable on storage.

A crystalline form of Compound I can be used to modulate/improve the physicochemical properties of the compound, including but not limited to solid state properties (e.g., crystallinity, hygroscopicity, melting point, or hydration), pharmaceutical properties (e.g., solubility/dissolution rate, stability, or compatibility), as well as crystallization characteristics (e.g., purity, yield, or morphology).

In one aspect, the invention features a crystalline form of Compound I which has characteristic peaks in the X-ray powder diffraction (XRPD) pattern as shown in FIG. 1 that is designated as crystalline Form A. Form A is an anhydrous form and a stable form at ambient temperature. By DVS, Form A appears to be non-hygroscopic, gaining just 0.1 wt % during adsorption to 95% RH and losing all of the gained moisture on desorption to 5% RH.

In one aspect, the crystalline Form A of Compound I has characteristic peaks in the powder X-ray diffraction (XRPD) pattern at values of two theta (° 2θ)/intensity (%) of 16.33 (42%), 17.10 (70%), 18.85 (78%), 20.62 (100%), 21.68 (71%), 22.11 (84%), 26.36 (40%).

In one aspect, the crystalline Form A of Compound I has characteristic peaks in the powder X-ray diffraction (XRPD) pattern at values of two theta (° 2θ)/intensity (%) of 7.04 (34%), 9.22 (35%), 9.64 (24%), 13.61 (23%), 14.28 (38%), 16.33 (42%), 17.10 (70%), 18.85 (78%), 19.37 (34%), 20.62 (100%), 21.68 (71%), 22.11 (84%), 24.71 (29%), 26.36 (40%).

In one aspect, the crystalline Form A of Compound I has characteristic peaks in the powder X-ray diffraction (XRPD) pattern at values of two theta (° 2θ)/intensity (%) of 7.04 (34%), 9.22 (35%), 9.64 (24%), 12.89 (11%), 13.61 (23%), 14.28 (38%), 16.33 (42%), 16.78 (21%), 17.10 (70%), 17.46 (11%), 18.03 (18%), 18.52 (19%), 18.85 (78%), 19.37 (34%), 19.68 (13%), 20.16 (14%), 20.62 (100%), 21.21 (10%), 21.68 (71%), 22.11 (84%), 22.66 (15%), 22.98 (26%), 23.15 (24%), 24.71 (29%), 25.38 (16%), 25.97 (17%), 26.36 (40%), and 28.83 (11%). The characteristic peaks can alternatively be represented as (20): 7.0, 9.2, 9.6, 12.9, 13.6, 14.3, 16.3, 16.8, 17.1, 17.5, 18.0, 18.5, 18.9, 19.4, 19.7, 20.2, 20.6, 21.2, 21.7, 22.1, 22.7, 23.0, 23.2, 24.7, 25.4, 26.0, 26.4, and 28.8.

In one aspect, the crystalline Form A of Compound I has characteristic peaks in the powder X-ray diffraction (XRPD) pattern at values of two theta (° 2θ)/intensity (%) of 7.04 (34%), 9.22 (35%), 9.64 (24%), 11.07 (9%), 11.43 (4%), 12.39 (6%), 12.89 (11%), 13.61 (23%), 14.28 (38%), 16.33 (42%), 16.78 (21%), 17.10 (70%), 17.46 (11%), 18.03 (18%), 18.52 (19%), 18.85 (78%), 19.37 (34%), 19.68 (13%), 19.89 (6%), 20.16 (14%), 20.62 (100%), 21.21 (10%), 21.68 (71%), 22.11 (84%), 22.66 (15%), 22.98 (26%), 23.15 (24%), 24.71 (29%), 24.94 (5%), 25.38 (16%), 25.97 (17%), 26.36 (40%), 26.80 (3%), 27.45 (8%), 27.92 (8%), 28.12 (6%), 28.27 (6%), 28.54 (8%), 28.83 (11%), and 29.40 (5%). The characteristic peaks can alternatively be represented as (20): 7.0, 9.2, 9.6, 11.1, 11.4, 12.4, 12.9, 13.6, 14.3, 16.3, 16.8, 17.1, 17.5, 18.0, 18.5, 18.9, 19.4, 19.7, 19.9, 20.2, 20.6, 21.2, 21.7, 22.1, 22.7, 23.0, 23.2, 24.7, 25.0, 25.4, 26.0, 26.4, 26.8, 27.5, 27.9, 28.1, 28.3, 28.5, 28.8, and 29.4.

In one aspect, the crystalline Form A of Compound I has characteristic peaks in the powder X-ray diffraction (XRPD) pattern at values of two theta (° 2θ) of 7.04±0.20, 9.22±0.20, 9.64±0.20, 12.89±0.20, 13.61±0.20, 14.28±0.20, 16.33±0.20, 16.78±0.20, 17.10±0.20, 17.46±0.20, 18.03±0.20, 18.52±0.20, 18.85±0.20, 19.37±0.20, 19.68±0.20, 20.16±0.20, 20.62±0.20, 21.21±0.20, 21.68±0.20, 22.11±0.20, 22.66±0.20, 22.98±0.20, 23.15±0.20, 24.71±0.20, 25.38±0.20, 25.97±0.20, 26.36±0.20, and 28.83±0.20.

In one aspect, the crystalline Form A of Compound I has characteristic peaks in the XRPD pattern at values of two theta (° 2θ) of 7.04±0.20, 9.22±0.20, 9.64±0.20, 11.07±0.20, 11.43±0.20, 12.39±0.20, 12.89±0.20, 13.61±0.20, 14.28±0.20, 16.33±0.20, 16.78±0.20, 17.10±0.20, 17.46±0.20, 18.03±0.20, 18.52±0.20, 18.85±0.20, 19.37±0.20, 19.68±0.20, 19.89±0.20, 20.16±0.20, 20.62±0.20, 21.21±0.20, 21.68±0.20, 22.11±0.20, 22.66±0.20, 22.98±0.20, 23.15±0.20, 24.71±0.20, 24.94±0.20, 25.38±0.20, 25.97±0.20, 26.36±0.20, 26.80±0.20, 27.45±0.20, 27.92±0.20, 28.12±0.20, 28.27±0.20, 28.54±0.20, 28.83±0.20, and 29.40±0.20.

In one embodiment, the crystalline Form A of Compound I has characteristic peaks in the X-ray powder diffraction (XRPD) pattern as shown in FIG. 1 (Form A) and Table 2 (Form A). The crystalline Form A of Compound I is preferably substantially pure. As used herein, the term “substantially pure”, when used in reference to a given crystalline form, refers to the crystalline form which is at least about 90% pure. This means that the crystalline form does not contain more than about 10% of any other form of Compound I. Preferably, the term “substantially pure” refers to a crystalline form of Compound I which is at least about 95% pure. This means that the crystalline form of Compound I does not contain more than about 5% of any other form of Compound I. More preferably, the term “substantially pure” refers to a crystalline form of Compound I which is at least about 97% pure. This means that the crystalline form of Compound I does not contain more than about 3% of any other form of Compound I.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 16.33, 17.10, 18.85, 20.62, 21.68, 22.11, 26.36, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.04, 9.22, 9.64, 13.61, 14.28, 16.33, 17.10, 18.85, 19.37, 20.62, 21.68, 22.11, 24.71, 26.36, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.04, 9.22, 9.64, 12.89, 13.61, 14.28, 16.33, 16.78, 17.10, 18.03, 18.52, 18.85, 19.37, 19.68, 20.16, 20.62, 21.68, 22.11, 22.98, 23.15, 24.71, 25.38, 25.97, 26.36, 28.83, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.04, 9.22, 9.64, 12.89, 13.61, 14.28, 16.33, 16.78, 17.10, 17.46, 18.03, 18.52, 18.85, 19.37, 19.68, 20.16, 20.62, 21.21, 21.68, 22.11, 22.66, 22.98, 23.15, 24.71, 25.38, 25.97, 26.36, and 28.83, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.0, 9.2, 9.6, 12.9, 13.6, 14.3, 16.3, 16.8, 17.1, 17.5, 18.0, 18.5, 18.9, 19.4, 19.7, 20.2, 20.6, 21.2, 21.7, 22.1, 22.7, 23.0, 23.2, 24.7, 25.4, 26.0, 26.4, and 28.8, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.04, 9.22, 9.64, 11.07, 11.43, 12.39, 12.89, 13.61, 14.28, 16.33, 16.78, 17.10, 17.46, 18.03, 18.52, 18.85, 19.37, 19.68, 19.89, 20.16, 20.62, 21.21, 21.68, 22.11, 22.66, 22.98, 23.15, 24.71, 24.94, 25.38, 25.97, 26.36, 26.80, 27.45, 27.92, 28.12, 28.27, 28.54, 28.83, and 29.40, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.0, 9.2, 9.6, 11.1, 11.4, 12.4, 12.9, 13.6, 14.3, 16.3, 16.8, 17.1, 17.5, 18.0, 18.5, 18.9, 19.4, 19.7, 19.9, 20.2, 20.6, 21.2, 21.7, 22.1, 22.7, 23.0, 23.2, 24.7, 25.0, 25.4, 26.0, 26.4, 26.8, 27.5, 27.9, 28.1, 28.3, 28.5, 28.8, and 29.4, and which is substantially pure. For example, the crystalline form used can be at least 90% pure, preferably at least 95% pure, or more preferably at least 97% pure.

In one aspect, the invention features a method for preparing Form A of Compound I comprising crystallizing Compound I from an appropriate solvent or solvent mixture including, but not limited to, ethanol, methanol, ethyl acetate, isopropyl acetate, acetone, dichloromethane (DCM), ethyl ether, toluene, methyl t-butyl ether (MTBE), heptane, hexanes, or mixture of two or more thereof.

In one aspect, the invention features a method for preparing Form A of Compound I comprising crystalizing a solution of Compound I in ethanol, ethyl acetate, isopropyl acetate; a mixture of ethanol and either acetone or DCM, or a mixture of DCM and MTBE.

In one aspect, the invention features a method for preparing Form A of Compound I by crystallizing the compound from a solution of DCM, followed by solvent replacement with Ethyl Acetate, Isopropyl acetate, or a mixture of MTBE and DCM, with or without seeding with Compound I of Form A.

In one aspect, the invention features a method for preparing Form A of Compound I by dissolving Compound I in EtOH, a mixture of acetone and ethanol or a mixture of DCM and ethanol and solvent switching to EtOH, with or without seeding with Compound I of Form A. A suitable single crystal of Form A of Compound I was selected and analyzed by single crystal X-ray diffractometry. The structure was determined and confirmed as described in Examples 1a, 1b, and 1c, and indicated the consistent form and that the crystal was representative of the bulk powder. Table 2 is listed the peak positions and intensities in the XRPD data for Compound I Crystalline Form A.

TABLE 2
Observed XRPD Peaks for Compound I, Crystalline Form A

| 2θ (°) | d space (Å) | Intensity (%) |
|---|---|---|
| 7.04 ± 0.20 | 12.539 ± 0.356 | 34 |
| 9.22 ± 0.20 | 9.587 ± 0.208 | 35 |
| 9.64 ± 0.20 | 9.166 ± 0.190 | 24 |
| 11.07 ± 0.20 | 7.989 ± 0.144 | 9 |
| 11.43 ± 0.20 | 7.733 ± 0.135 | 4 |
| 12.39 ± 0.20 | 7.138 ± 0.115 | 6 |
| 12.89 ± 0.20 | 6.864 ± 0.106 | 11 |
| 13.61 ± 0.20 | 6.501 ± 0.095 | 23 |
| 14.28 ± 0.20 | 6.200 ± 0.086 | 38 |
| 16.33 ± 0.20 | 5.422 ± 0.066 | 42 |
| 16.78 ± 0.20 | 5.279 ± 0.062 | 21 |
| 17.10 ± 0.20 | 5.182 ± 0.060 | 70 |
| 17.46 ± 0.20 | 5.075 ± 0.058 | 11 |
| 18.03 ± 0.20 | 4.917 ± 0.054 | 18 |
| 18.52 ± 0.20 | 4.788 ± 0.051 | 19 |
| 18.85 ± 0.20 | 4.703 ± 0.049 | 78 |
| 19.37 ± 0.20 | 4.578 ± 0.047 | 34 |
| 19.68 ± 0.20 | 4.507 ± 0.045 | 13 |
| 19.89 ± 0.20 | 4.460 ± 0.044 | 6 |
| 20.16 ± 0.20 | 4.400 ± 0.043 | 14 |
| 20.62 ± 0.20 | 4.304 ± 0.041 | 100 |
| 21.21 ± 0.20 | 4.185 ± 0.039 | 10 |
| 21.68 ± 0.20 | 4.096 ± 0.037 | 71 |
| 22.11 ± 0.20 | 4.017 ± 0.036 | 84 |
| 22.66 ± 0.20 | 3.920 ± 0.034 | 15 |
| 22.98 ± 0.20 | 3.867 ± 0.033 | 26 |
| 23.15 ± 0.20 | 3.839 ± 0.033 | 24 |
| 24.71 ± 0.20 | 3.599 ± 0.029 | 29 |
| 24.94 ± 0.20 | 3.567 ± 0.028 | 5 |
| 25.38 ± 0.20 | 3.506 ± 0.027 | 16 |
| 25.97 ± 0.20 | 3.428 ± 0.026 | 17 |
| 26.36 ± 0.20 | 3.378 ± 0.025 | 40 |
| 26.80 ± 0.20 | 3.324 ± 0.024 | 3 |
| 27.45 ± 0.20 | 3.246 ± 0.023 | 8 |
| 27.92 ± 0.20 | 3.193 ± 0.022 | 8 |
| 28.12 ± 0.20 | 3.170 ± 0.022 | 6 |
| 28.27 ± 0.20 | 3.154 ± 0.022 | 6 |
| 28.54 ± 0.20 | 3.125 ± 0.021 | 8 |
| 28.83 ± 0.20 | 3.094 ± 0.021 | 11 |
| 29.40 ± 0.20 | 3.035 ± 0.020 | 5 |

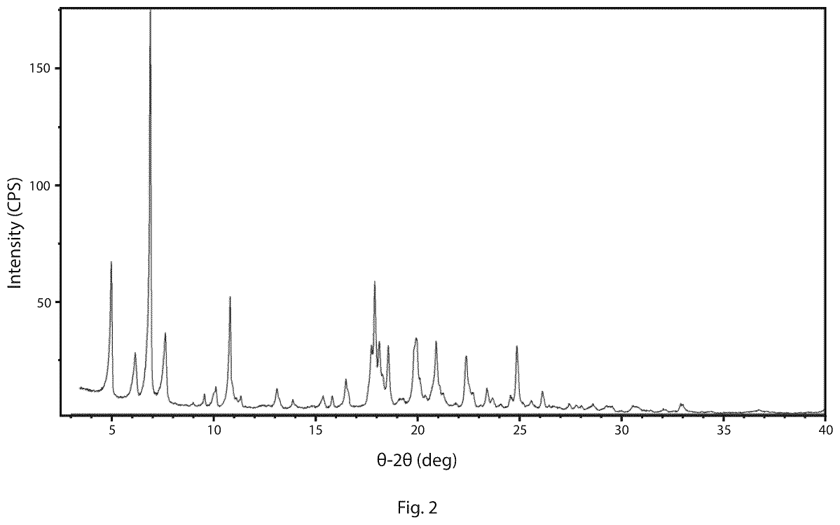

In another aspect, the invention features a crystalline form of Compound I which has characteristic peaks in the X-ray powder diffraction (XRPD) pattern as shown in FIG. 2, a solid form from methanol/toluene slurry, designated Material B, a possible toluene solvate material.

Slow evaporation of a solution of Compound I in methanol/toluene (1:4 v/v) generated a mixture of Form A and Material B, and further slurrying such mixture in this solvent system yielded Material B. A slurry of Form A in this solvent system resulted in only Form A. In another slurry experiment in this solvent system, Form A was seeded with a small amount of Material B and the sample was stirred for a longer period (more than twice as many days as in the slurry of Form A only). A mixture of Form A and Material B resulted, in which Form A was the predominant material. These results suggest that the conversion from Form A to Material B is slow. The possible reason is that Material B is a toluene solvate based on experimental conditions.

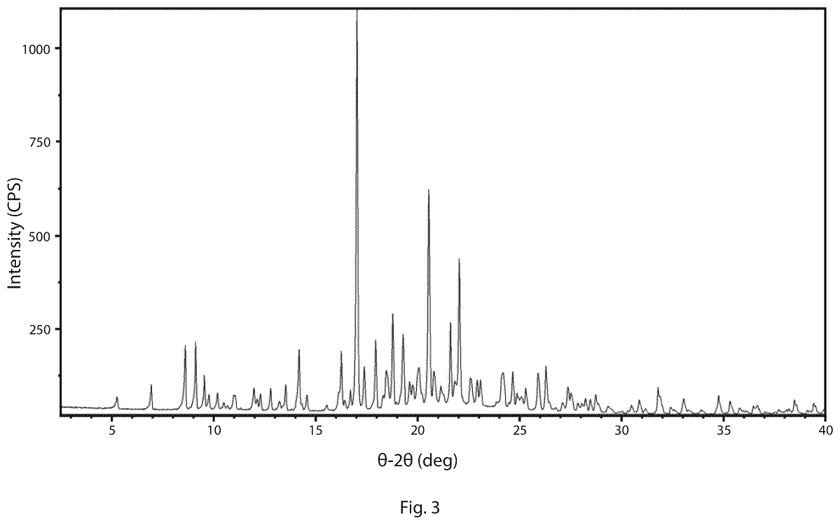

In yet another aspect, the invention features a crystalline form of Compound I that is a mixture of Form A and Material C of Compound I, which has characteristic peaks in the X-ray powder diffraction (XRPD) pattern as shown in FIG. 3.

Material C was obtained from saturated ethanol solutions of Compound I by cooling from 60° C., followed by evaporation at ambient temperature, or by evaporation at 60° C. Various temperature conditions were examined; however, Material C was only observed when the ethanol solution was prepared at 60° C. In addition, a relatively larger amount of Material C was obtained when the solution was evaporated at this temperature.

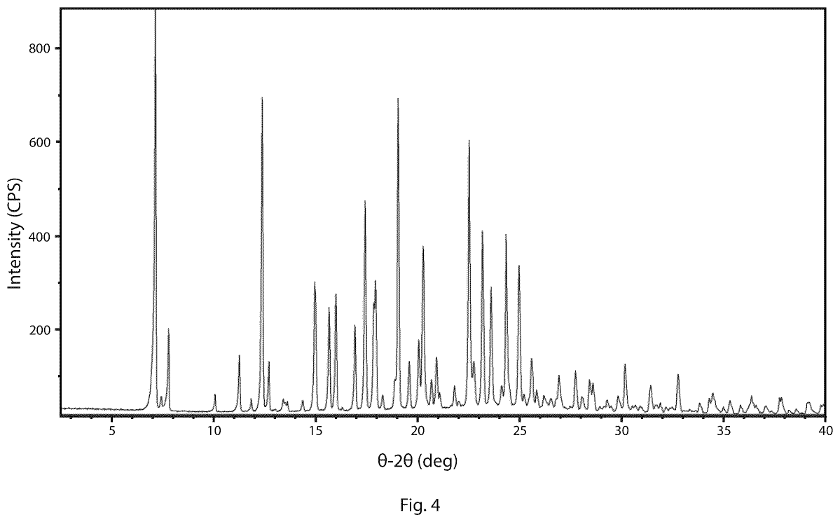

In yet another aspect, the invention features a crystalline form of Compound I which has characteristic peaks in the X-ray powder diffraction (XRPD) pattern as shown in FIG. 4 and peak list [2θ(°)/intensity (%)] as shown in Table 3, designated as Form D.

In one aspect, the crystalline Form D of Compound I has characteristic peaks in the powder X-ray diffraction (XRPD) pattern at values of two theta (° 2θ)/intensity (%) of 7.19 (100%), 12.42 (83%), 17.47 (55%), 19.08 (78%), 20.31 (45%), 22.57 (70%), 23.22 (49%), 24.39 (47%), 25.02 (40%).

In one aspect, the crystalline Form D of Compound I has characteristic peaks in the powder X-ray diffraction (XRPD) pattern at values of two theta (° 2θ)/intensity (%) of 7.19 (100%), 7.83 (23%), 12.42 (83%), 14.99 (36%), 15.70 (29%), 16.04 (33%), 16.97 (25%), 17.47 (55%), 17.89 (30%), 17.97 (36%), 19.08 (78%), 20.31 (45%), 22.57 (70%), 23.22 (49%), 23.64 (34%), 24.39 (47%), 25.02 (40%).

In one aspect, the crystalline Form D of Compound I has characteristic peaks in the powder X-ray diffraction (XRPD) pattern at values of two theta (° 2θ)/intensity (%) of 7.19 (100%), 7.48 (7%), 7.83 (23%), 10.11 (8%), 11.29 (17%), 11.88 (6%), 12.42 (83%), 12.74 (16%), 13.45 (6%), 13.54 (5%), 13.65 (6%), 14.41 (6%), 14.99 (36%), 15.70 (29%), 16.04 (33%), 16.36 (4%), 16.97 (25%), 17.47 (55%), 17.89 (30%), 17.97 (36%), 18.33 (7%), 18.91 (11%), 19.08 (78%), 19.63 (16%), 20.10 (21%), 20.31 (45%), 20.72 (11%), 20.97 (17%), 21.12 (8%), 21.85 (10%), 22.06 (6%), 22.57 (70%), 22.80 (16%), 23.22 (49%), 23.64 (34%), 24.16 (10%), 24.39 (47%), 25.02 (40%), 25.25 (7%), 25.63 (17%), 25.89 (8%), 26.24 (7%), 26.56 (6%), 26.97 (12%), 27.78 (13%), 28.12 (7%), 28.49 (11%), and 28.64 (10%).

In one embodiment, the present invention provides crystalline Form D of Compound I which has characteristic peaks in the X-ray powder diffraction (XRPD) pattern as shown in FIG. 4 (Form D) and Table 3 (Form D) and which is substantially pure. As used herein, the term “substantially pure”, when used in reference to a given crystalline form, refers to the crystalline form which is at least about 90% pure. This means that the crystalline form does not contain more than about 10% of any other form of Compound I. Preferably, the term “substantially pure” refers to a crystalline form of Compound I which is at least about 95% pure. This means that the crystalline form of Compound I does not contain more than about 5% of any other form of Compound I. More preferably, the term “substantially pure” refers to a crystalline form of Compound I which is at least about 97% pure. This means that the crystalline form of Compound I does not contain more than about 3% of any other form of Compound I.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.19, 12.42, 17.47, 19.08, 20.31, 22.57, 23.22, 24.39, 25.02, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.19, 7.83, 12.42, 14.99, 15.70, 16.04, 16.97, 17.47, 17.89, 17.97, 19.08, 20.31, 22.57, 23.22, 23.64, 24.39, 25.02, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.19, 7.83, 12.42, 12.74, 14.99, 15.70, 16.04, 16.97, 17.47, 17.89, 17.97, 19.08, 19.63, 20.1, 20.31, 20.97, 22.57, 22.80, 23.22, 23.64, 24.39, 25.02, 25.63, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.19, 7.48, 7.83, 10.1, 11.29, 11.88, 12.42, 12.74, 13.45, 13.54, 13.65, 14.41, 14.99, 15.70, 16.04, 16.36, 16.97, 17.47, 17.89, 17.97, 18.33, 18.91, 19.08, 19.63, 20.10, 20.31, 20.72, 20.97, 21.12, 21.85, 22.06, 22.57, 22.80, 23.22, 23.64, 24.16, 24.39, 25.02, 25.25, 25.63, 25.89, 26.24, 26.56, 26.97, 27.78, 28.12, 28.49, and 28.64, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.2, 7.5, 7.8, 10.0, 11.3, 11.9, 12.4, 12.7, 13.5, 13.5, 13.7, 14.4, 15.0, 15.7, 16.0, 16.4, 17.0, 17.5, 17.9, 18.0, 18.3, 18.9, 19.1, 19.6, 20.1, 20.3, 20.7, 21.0, 21.1, 21.9, 22.1, 22.6, 22.8, 23.2, 23.6, 24.2, 24.4, 25.0, 25.3, 25.6, 25.9, 26.2, 26.6, 27.0, 27.8, 28.1, 28.49, and 28.6, and which is substantially pure. For example, the crystalline form used can be at least 90% pure, preferably at least 95% pure, or more preferably at least 97% pure.

TABLE 3
Observed XRPD Peaks for Compound I Crystalline Form D

| 2θ (°) | d space (Å) | Intensity (%) |
|---|---|---|
| 7.19 ± 0.20 | 12.285 ± 0.341 | 100 |
| 7.48 ± 0.20 | 11.809 ± 0.315 | 7 |
| 7.83 ± 0.20 | 11.282 ± 0.288 | 23 |
| 10.11 ± 0.20 | 8.742 ± 0.172 | 8 |
| 11.29 ± 0.20 | 7.831 ± 0.138 | 17 |
| 11.88 ± 0.20 | 7.443 ± 0.125 | 6 |
| 12.42 ± 0.20 | 7.121 ± 0.114 | 83 |
| 12.74 ± 0.20 | 6.943 ± 0.109 | 16 |
| 13.45 ± 0.20 | 6.578 ± 0.097 | 6 |
| 13.54 ± 0.20 | 6.534 ± 0.096 | 5 |
| 13.65 ± 0.20 | 6.482 ± 0.095 | 6 |
| 14.41 ± 0.20 | 6.142 ± 0.085 | 6 |
| 14.99 ± 0.20 | 5.905 ± 0.078 | 36 |
| 15.70 ± 0.20 | 5.640 ± 0.071 | 29 |
| 16.04 ± 0.20 | 5.521 ± 0.068 | 33 |
| 16.36 ± 0.20 | 5.414 ± 0.066 | 4 |
| 16.97 ± 0.20 | 5.221 ± 0.061 | 25 |
| 17.47 ± 0.20 | 5.072 ± 0.058 | 55 |
| 17.89 ± 0.20 | 4.954 ± 0.055 | 30 |
| 17.97 ± 0.20 | 4.932 ± 0.054 | 36 |
| 18.33 ± 0.20 | 4.836 ± 0.052 | 7 |
| 18.91 ± 0.20 | 4.689 ± 0.049 | 11 |
| 19.08 ± 0.20 | 4.648 ± 0.048 | 78 |
| 19.63 ± 0.20 | 4.519 ± 0.046 | 16 |
| 20.10 ± 0.20 | 4.414 ± 0.043 | 21 |
| 20.31 ± 0.20 | 4.369 ± 0.043 | 45 |
| 20.72 ± 0.20 | 4.283 ± 0.041 | 11 |
| 20.97 ± 0.20 | 4.233 ± 0.040 | 17 |
| 21.12 ± 0.20 | 4.203 ± 0.039 | 8 |
| 21.85 ± 0.20 | 4.064 ± 0.037 | 10 |
| 22.06 ± 0.20 | 4.026 ± 0.036 | 6 |
| 22.57 ± 0.20 | 3.936 ± 0.034 | 70 |
| 22.80 ± 0.20 | 3.897 ± 0.034 | 16 |
| 23.22 ± 0.20 | 3.828 ± 0.033 | 49 |
| 23.64 ± 0.20 | 3.761 ± 0.031 | 34 |
| 24.16 ± 0.20 | 3.681 ± 0.030 | 10 |
| 24.39 ± 0.20 | 3.647 ± 0.029 | 47 |
| 25.02 ± 0.20 | 3.556 ± 0.028 | 40 |
| 25.25 ± 0.20 | 3.524 ± 0.027 | 7 |
| 25.63 ± 0.20 | 3.473 ± 0.027 | 17 |
| 25.89 ± 0.20 | 3.439 ± 0.026 | 8 |
| 26.24 ± 0.20 | 3.394 ± 0.025 | 7 |
| 26.56 ± 0.20 | 3.353 ± 0.025 | 6 |
| 26.97 ± 0.20 | 3.303 ± 0.024 | 12 |
| 27.78 ± 0.20 | 3.209 ± 0.023 | 13 |
| 28.12 ± 0.20 | 3.171 ± 0.022 | 7 |
| 28.49 ± 0.20 | 3.131 ± 0.022 | 11 |
| 28.64 0.20 | 3.114 ± 0.021 | 10 |

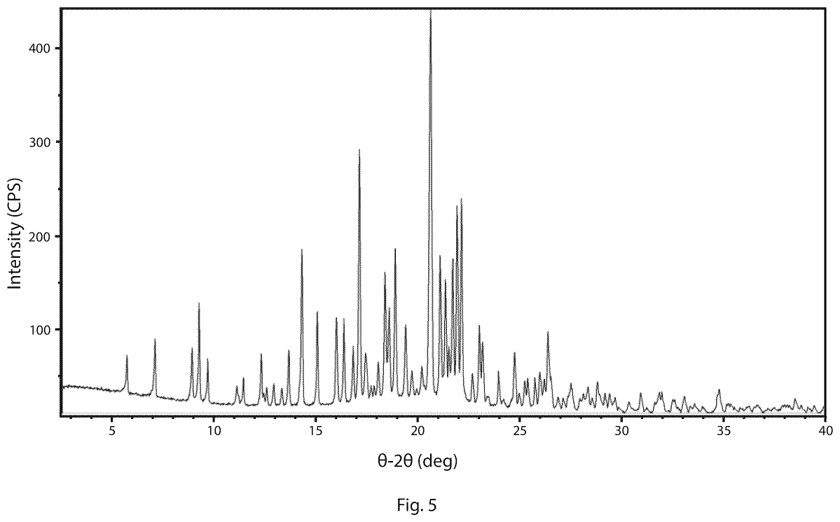

In yet another aspect, the invention features a crystalline form of Compound I which has characteristic peaks in the X-ray powder diffraction (XRPD) pattern as shown in FIG. 5 that is a mixture of Form A and Material E.

Since Material E was only observed in mixtures with Form A, the corresponding XRPD patterns could not be directly indexed. The 1H-NMR spectrum of an air-dried Form A/Material E mixture is predominantly consistent with Compound I. The spectrum shows ˜0.1 mole of tetrahydrofuran per mole of Compound I, which could be consistent with the suspected solvated material based on Pawley refinement. However, since the sample is a mixture, the actual amount of tetrahydrofuran in Material E cannot be calculated based on the 1H-NMR data. In addition, the possibility of residual tetrahydrofuran in the sample cannot be ruled out. Therefore, additional characterization is necessary to determine the nature of Material E.

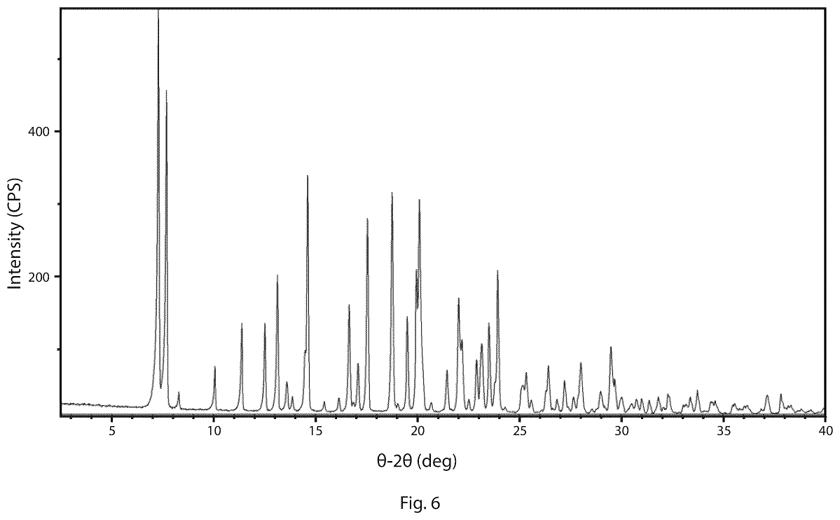

In yet another aspect, the invention features a crystalline form of Compound I which has characteristic peaks in the X-ray powder diffraction (XRPD) pattern as shown in FIG. 6 and peak list [2θ(°)/intensity (%)] as shown in Table 4, designates as Form F.

In one aspect, the crystalline Form F of Compound I has characteristic peaks in the powder X-ray diffraction (XRPD) pattern at values of two theta (° 2θ)/intensity (%) of 7.29 (100%), 7.69 (79%), 14.63 (59%), 17.56 (49%), 18.77 (55%), 20.12 (54%).

In one aspect, the crystalline Form F of Compound I has characteristic peaks in the powder X-ray diffraction (XRPD) pattern at values of two theta (° 2θ)/intensity (%) of 7.29 (100%), 7.69 (79%), 11.38 (23%), 11.52 (23%), 13.13 (35%), 14.63 (59%), 16.66 (28%), 17.56 (49%), 18.77 (55%), 19.52 (25%), 19.97 (37%), 20.12 (54%), 22.04 (30%), 23.97 (37%).

In one aspect, the crystalline Form F of Compound I has characteristic peaks in the powder X-ray diffraction (XRPD) pattern at values of two theta (° 2θ)/intensity (%) of 7.29 (100%), 7.69 (79%), 8.31 (7%), 10.06 (13%), 11.38 (23%), 12.52 (23%), 13.13 (35%), 13.60 (9%), 13.87 (6%), 14.49 (17%), 14.63 (59%), 15.43 (4%), 16.16 (5%), 16.66 (28%), 16.87 (4%), 17.09 (14%), 17.56 (49%), 18.77 (55%), 19.04 (4%), 19.52 (25%), 19.97 (37%), 20.12 (54%), 20.68 (4%), 21.47 (12%), 22.04 (30%), 22.20 (19%), 22.53 (5%), 22.91 (15%), 23.17 (18%), 23.54 (23%), 23.81 (9%), 23.97 (37%), 24.31 (3%), 25.17 (8%), 25.36 (11%), 25.60 (5%), 26.11 (2%), 26.32 (7%), 26.44 (13%), 26.86 (5%), 27.23 (9%), 27.68 (6%), 28.05 (14%), 28.58 (3%), 29.01 (7%), 29.52 (18%), and 29.69 (10%).

In one embodiment, the crystalline Form F of Compound I has characteristic peaks in the X-ray powder diffraction (XRPD) pattern as shown in FIG. 6 (Form F) and Table 4 (Form F) and which is substantially pure. As used herein, the term “substantially pure”, when used in reference to a given crystalline form, refers to the crystalline form which is at least about 90% pure. This means that the crystalline form does not contain more than about 10% of any other form of Compound I. Preferably, the term “substantially pure” refers to a crystalline form of Compound I which is at least about 95% pure. This means that the crystalline form of Compound I does not contain more than about 5% of any other form of Compound I. More preferably, the term “substantially pure” refers to a crystalline form of Compound I which is at least about 97% pure. This means that the crystalline form of Compound I does not contain more than about 3% of any other form of Compound I.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.29, 7.69, 14.63, 17.56, 18.77, 20.12, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.29, 7.69, 11.38, 11.52, 13.13, 14.63, 16.66, 17.56, 18.77, 19.52, 19.97, 20.12, 22.04, 23.97, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.29, 7.69, 11.38, 11.52, 13.13, 14.49, 14.63, 16.66, 17.56, 18.77, 19.52, 19.97, 20.12, 22.04, 22.20, 22.91, 23.54, 23.97, 29.52, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.29, 7.69, 8.31, 10.06, 11.38, 12.52, 13.13, 13.60, 13.87, 14.49, 14.63, 15.43, 16.16, 16.66, 16.87, 17.09, 17.56, 18.77, 19.04, 19.52, 19.97, 20.12, 20.68, 21.47, 22.04, 22.20, 22.53, 22.91, 23.17, 23.54, 23.81, 23.97, 24.31, 25.17, 25.36, 25.60, 26.11, 26.32, 26.44, 26.86, 27.23, 27.68, 28.05, 28.58, 29.01, 29.52, and 29.69, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.3, 7.7, 8.3, 10.1, 11.4, 12.5, 13.1, 13.6, 13.9, 14.5, 14.6, 15.4, 16.2, 16.7, 16.9, 17.1, 17.6, 18.8, 19.0, 19.5, 20.0, 20.1, 20.7, 21.5, 22.0, 22.2, 22.5, 22.9, 23.2, 23.5, 23.8, 24.0, 24.3, 25.2, 25.4, 25.6, 26.1, 26.3, 26.4, 26.9, 27.2, 27.7, 28.1, 28.6, 29.0, 29.5, and 29.7, and which is substantially pure. For example, the crystalline form used can be at least 90% pure, preferably at least 95% pure, or more preferably at least 97% pure.

TABLE 4
Observed XRPD Peaks for Compound I Crystalline Form F

| 2θ (°) | d space (Å) | Intensity (%) |
|---|---|---|
| 7.29 ± 0.20 | 12.116 ± 0.332 | 100 |
| 7.69 ± 0.20 | 11.491 ± 0.299 | 79 |
| 8.31 ± 0.20 | 10.635 ± 0.256 | 7 |
| 10.06 ± 0.20 | 8.784 ± 0.174 | 13 |
| 11.38 ± 0.20 | 7.770 ± 0.136 | 23 |
| 12.52 ± 0.20 | 7.063 ± 0.112 | 23 |
| 13.13 ± 0.20 | 6.736 ± 0.102 | 35 |
| 13.60 ± 0.20 | 6.507 ± 0.095 | 9 |
| 13.87 ± 0.20 | 6.380 ± 0.092 | 6 |
| 14.49 ± 0.20 | 6.108 ± 0.084 | 17 |
| 14.63 ± 0.20 | 6.049 ± 0.082 | 59 |
| 15.43 ± 0.20 | 5.736 ± 0.074 | 4 |
| 16.16 ± 0.20 | 5.482 ± 0.067 | 5 |
| 16.66 ± 0.20 | 5.317 ± 0.063 | 28 |
| 16.87 ± 0.20 | 5.253 ± 0.062 | 4 |
| 17.09 ± 0.20 | 5.183 ± 0.060 | 14 |
| 17.56 ± 0.20 | 5.047 ± 0.057 | 49 |
| 18.77 ± 0.20 | 4.723 ± 0.050 | 55 |
| 19.04 ± 0.20 | 4.656 ± 0.048 | 4 |
| 19.52 ± 0.20 | 4.544 ± 0.046 | 25 |
| 19.97 ± 0.20 | 4.443 ± 0.044 | 37 |
| 20.12 ± 0.20 | 4.411 ± 0.043 | 54 |
| 20.68 ± 0.20 | 4.292 ± 0.041 | 4 |
| 21.47 ± 0.20 | 4.136 ± 0.038 | 12 |
| 22.04 ± 0.20 | 4.031 ± 0.036 | 30 |
| 22.20 ± 0.20 | 4.001 ± 0.036 | 19 |
| 22.53 ± 0.20 | 3.943 ± 0.035 | 5 |
| 22.91 ± 0.20 | 3.878 ± 0.033 | 15 |
| 23.17 ± 0.20 | 3.836 ± 0.033 | 18 |
| 23.54 ± 0.20 | 3.777 ± 0.032 | 23 |
| 23.81 ± 0.20 | 3.733 ± 0.031 | 9 |
| 23.97 ± 0.20 | 3.710 ± 0.031 | 37 |
| 24.31 ± 0.20 | 3.658 ± 0.030 | 3 |
| 25.17 ± 0.20 | 3.535 ± 0.028 | 8 |
| 25.36 ± 0.20 | 3.509 ± 0.027 | 11 |
| 25.60 ± 0.20 | 3.477 ± 0.027 | 5 |
| 26.11 ± 0.20 | 3.410 ± 0.026 | 2 |
| 26.32 ± 0.20 | 3.384 ± 0.025 | 7 |
| 26.44 ± 0.20 | 3.368 ± 0.025 | 13 |
| 26.86 ± 0.20 | 3.317 ± 0.024 | 5 |
| 27.23 ± 0.20 | 3.272 ± 0.024 | 9 |
| 27.68 ± 0.20 | 3.220 ± 0.023 | 6 |
| 28.05 ± 0.20 | 3.179 ± 0.022 | 14 |
| 28.58 ± 0.20 | 3.121 ± 0.021 | 3 |
| 29.01 ± 0.20 | 3.075 ± 0.021 | 7 |
| 29.52 ± 0.20 | 3.024 ± 0.020 | 18 |
| 29.69 ± 0.20 | 3.006 ± 0.020 | 10 |

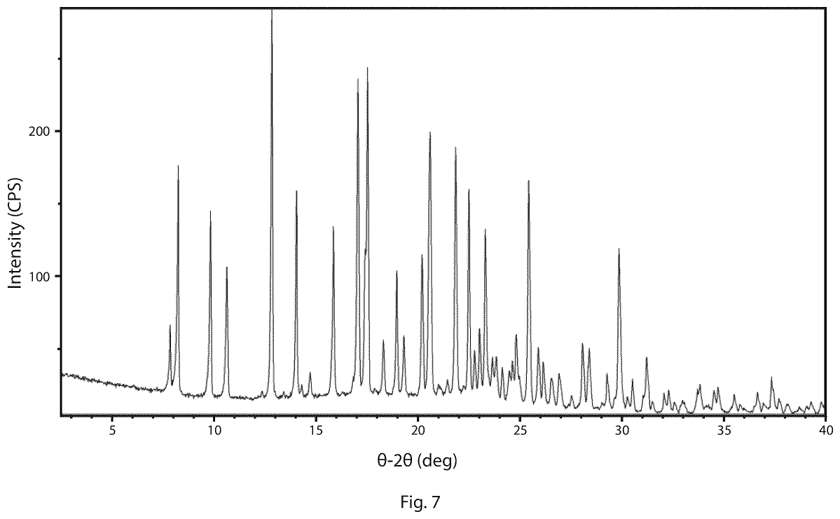

In yet another aspect, the invention features a crystalline form of Compound I which has characteristic peaks in the X-ray powder diffraction (XRPD) pattern as shown in FIG. 7 and peak list [2θ(°)/intensity (%)] as shown in Table 5, designated as Form G.

In one aspect, the crystalline Form G of Compound I has characteristic peaks in the powder X-ray diffraction (XRPD) pattern at values of two theta [2θ(°)/intensity (%)] of 8.27 (62%), 9.86 (51%), 12.88 (100%), 14.08 (57%), 15.90 (48%), 17.10 (85%), 17.44 (42%), 17.57 (85%), 20.25 (41%), 20.63 (71%), 21.90 (67%), 22.55 (57%), 23.35 (47%), 25.48 (60%), 29.92 (43%).

In one aspect, the crystalline Form G of Compound I has characteristic peaks in the powder X-ray diffraction (XRPD) pattern at values of two theta 20) (°)/intensity (%) of 8.27 (62%), 9.86 (51%), 10.67 (39%), 12.88 (100%), 14.08 (57%), 15.90 (48%), 17.10 (85%), 17.44 (42%), 17.57 (85%), 19.00 (38%), 20.25 (41%), 20.63 (71%), 21.90 (67%), 22.55 (57%), 23.35 (47%), 25.48 (60%), 29.92 (43%).

In one aspect, the crystalline Form G of Compound I has characteristic peaks in the powder X-ray diffraction (XRPD) pattern at values of two theta 2θ(°)/intensity (%) of 7.88 (24%), 8.27 (62%), 9.86 (51%), 10.67 (39%), 12.88 (100%), 14.08 (57%), 14.34 (10%), 14.75 (13%), 15.90 (48%), 17.10 (85%), 17.44 (42%), 17.57 (85%), 18.35 (21%), 19.00 (38%), 19.36 (22%), 20.25 (41%), 20.63 (71%), 21.48 (11%), 21.90 (67%), 22.55 (57%), 22.82 (18%), 23.07 (24%), 23.35 (47%), 23.69 (17%), 23.89 (17%), 24.18 (14%), 24.53 (14%), 24.68 (16%), 24.87 (22%), 25.48 (60%), 25.95 (19%), 26.21 (15%), 26.60 (12%), 26.97 (13%), 27.60 (7%), 28.13 (20%), 28.46 (19%), 29.33 (13%), and 29.92 (43%).

In one embodiment, the crystalline Form G of Compound I has characteristic peaks in the X-ray powder diffraction (XRPD) pattern as shown in FIG. 7 (Form G) and Table 5 (Form G) and which is substantially pure. As used herein, the term “substantially pure”, when used in reference to a given crystalline form, refers to the crystalline form which is at least about 90% pure. This means that the crystalline form does not contain more than about 10% of any other form of Compound I. Preferably, the term “substantially pure” refers to a crystalline form of Compound I which is at least about 95% pure. This means that the crystalline form of Compound I does not contain more than about 5% of any other form of Compound I. More preferably, the term “substantially pure” refers to a crystalline form of Compound I which is at least about 97% pure. This means that the crystalline form of Compound I does not contain more than about 3% of any other form of Compound I.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 8.27, 9.86, 12.88, 14.08, 15.90, 17.10, 17.44, 17.57, 20.25, 20.63, 21.90, 22.55, 23.35, 25.48, 29.92, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 8.27, 9.86, 10.67, 12.88, 14.08, 15.90, 17.10, 17.44, 17.57, 19.00, 20.25, 20.63, 21.90, 22.55, 23.35, 25.48, 29.92, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.88, 8.27, 9.86, 10.67, 12.88, 14.08, 15.90, 17.10, 17.44, 17.57, 18.35, 19.00, 19.36, 20.25, 20.63, 21.90, 22.55, 22.82, 23.07, 23.35, 23.89, 24.87, 25.48, 25.95, 26.21, 28.13, 28.46, 29.92, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.88, 8.27, 9.86, 10.67, 12.88, 14.08, 14.34, 14.75, 15.90, 17.10, 17.44, 17.57, 18.35, 19.00, 19.36, 20.25, 20.63, 21.48, 21.90, 22.55, 22.82, 23.07, 23.35, 23.69, 23.89, 24.18, 24.53, 24.68, 24.87, 25.48, 25.95, 26.21, 26.60, 26.97, 27.60, 28.13, 28.46, 29.33, and 29.92, and which is substantially pure. For example, the crystalline form used can be at least 90% pure, preferably at least 95% pure, or more preferably at least 97% pure.

In another embodiment, a method of the invention described herein (including any method described in any aspect, embodiment, example or preference) uses a crystalline form of Compound I which has characteristic peaks in the X-ray powder diffraction (XRPD) pattern at values of two theta (° 2θ) of 7.9, 8.3, 9.9, 10.7, 12.9, 14.1, 14.3, 14.8, 15.9, 17.1, 17.4, 17.6, 18.4, 19.00, 19.4, 20.3, 20.6, 21.5, 21.9, 22.6, 22.8, 23.1, 23.4, 23.7, 23.9, 24.2, 24.5, 24.7, 24.9, 25.5, 26.0, 26.2, 26.6, 27.0, 27.6, 28.1, 28.5, 29.3, and 29.9, and which is substantially pure. For example, the crystalline form used can be at least 90% pure, preferably at least 95% pure, or more preferably at least 97% pure.

TABLE 5
Observed XRPD Peaks for Compound I Crystalline Form G

| 2θ (°) | d space (Å) | Intensity (%) |
|---|---|---|
| 7.88 ± 0.20 | 11.211 ± 0.284 | 24 |
| 8.27 ± 0.20 | 10.682 ± 0.258 | 62 |
| 9.86 ± 0.20 | 8.964 ± 0.181 | 51 |
| 10.67 ± 0.20 | 8.284 ± 0.155 | 39 |
| 12.88 ± 0.20 | 6.867 ± 0.106 | 100 |
| 14.08 ± 0.20 | 6.286 ± 0.089 | 57 |
| 14.34 ± 0.20 | 6.173 ± 0.086 | 10 |
| 14.75 ± 0.20 | 5.999 ± 0.081 | 13 |
| 15.90 ± 0.20 | 5.571 ± 0.070 | 48 |
| 17.10 ± 0.20 | 5.182 ± 0.060 | 85 |
| 17.44 ± 0.20 | 5.080 ± 0.058 | 42 |
| 17.57 ± 0.20 | 5.044 ± 0.057 | 85 |
| 18.35 ± 0.20 | 4.831 ± 0.052 | 21 |
| 19.00 ± 0.20 | 4.666 ± 0.049 | 38 |
| 19.36 ± 0.20 | 4.582 ± 0.047 | 22 |
| 20.25 ± 0.20 | 4.382 ± 0.043 | 41 |
| 20.63 ± 0.20 | 4.301 ± 0.041 | 71 |
| 21.48 ± 0.20 | 4.134 ± 0.038 | 11 |
| 21.90 ± 0.20 | 4.056 ± 0.037 | 67 |
| 22.55 ± 0.20 | 3.940 ± 0.035 | 57 |
| 22.82 ± 0.20 | 3.893 ± 0.034 | 18 |
| 23.07 ± 0.20 | 3.851 ± 0.033 | 24 |
| 23.35 ± 0.20 | 3.806 ± 0.032 | 47 |
| 23.69 ± 0.20 | 3.752 ± 0.031 | 17 |
| 23.89 ± 0.20 | 3.722 ± 0.031 | 17 |
| 24.18 ± 0.20 | 3.677 ± 0.030 | 14 |
| 24.53 ± 0.20 | 3.626 ± 0.029 | 14 |
| 24.68 ± 0.20 | 3.604 ± 0.029 | 16 |
| 24.87 ± 0.20 | 3.577 ± 0.028 | 22 |
| 25.48 ± 0.20 | 3.492 ± 0.027 | 60 |
| 25.95 ± 0.20 | 3.430 ± 0.026 | 19 |
| 26.21 ± 0.20 | 3.398 ± 0.025 | 15 |
| 26.60 ± 0.20 | 3.348 ± 0.025 | 12 |
| 26.97 ± 0.20 | 3.304 ± 0.024 | 13 |
| 27.60 ± 0.20 | 3.230 ± 0.023 | 7 |
| 28.13 ± 0.20 | 3.170 ± 0.022 | 20 |
| 28.46 ± 0.20 | 3.134 ± 0.022 | 19 |
| 29.33 ± 0.20 | 3.042 ± 0.020 | 13 |
| 29.92 ± 0.20 | 2.984 ± 0.019 | 43 |

Form G of Compound I was initially observed when slurrying Forms A/F in ethyl acetate. Form G was obtained in mixture with Form A, regardless of slurry temperatures or solid wetness/isolation technique. The mixture appeared unchanged when vacuum dried at ambient temperature for 3 days, indicating that Form G is physically stable at ambient temperature. A single phase of Form G was generated by slurrying of Form Fin ethyl acetate. The XRPD pattern of Form G is shown in FIG. 7.

The ¹HNMR spectrum of the vacuum dried Form A/G mixture is predominantly consistent with Compound I. The spectrum shows only a trace amount of ethyl acetate, likely indicating residual solvent. This is consistent with the indexing results that Form G is likely an anhydrous/unsolvated material.

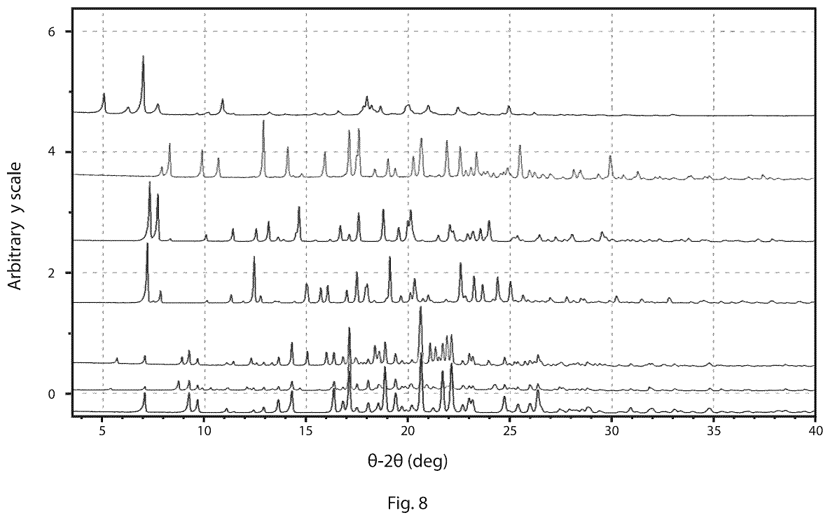

An overlay of XRPD patterns of all the observed crystalline solids, a total of seven (7) crystal forms or materials—A, B, C, D, E, F, and G, is shown in FIG. 8.

### EXAMPLES

Transmission Geometry: XRPD patterns were collected with a PANalytical X′Pert PRO MPD diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e) was analyzed to verify that the observed position of the Si 111 peak is consistent with the NIST-certified position. A specimen of the sample was sandwiched between 3-μm-thick films and analyzed in transmission geometry. A beam-stop, short anti-scatter extension, and anti-scatter knife edge were used to minimize the background generated by air. Soller slits for the incident and diffracted beams were used to minimize broadening from axial divergence. Diffraction patterns were collected using a scanning position-sensitive detector (X′Celerator) located 240 mm from the specimen and Data Collector software v. 2.2b.

Reflection Geometry: XRPD patterns were collected with a PANalytical X′Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Anti-scatter slits (SS) were used to minimize the background generated by air. Soller slits for the incident and diffracted beams were used to minimize broadening from axial divergence. Diffraction patterns were collected using a scanning position-sensitive detector (X′Celerator) located 240 mm from the sample and Data Collector software v. 2.2b.

The present invention features compositions comprising a crystalline form of Compound I of the invention. Any crystalline form described herein (including any crystalline form described in any aspect, embodiment or example) can be used to make a composition of the invention. Preferably, the crystalline form is substantially pure, such as at least 90% pure, preferably at least 95% pure, or more preferably at least 97% pure. In one embodiment, a composition of the invention comprises at least 5% by weight of a substantially pure crystalline form of the invention. In another embodiment, the composition of the invention comprises at least 10% by weight of a substantially pure crystalline form of the invention. In still another embodiment, a composition of the invention comprises at least 5% by weight of one or more crystalline forms of the invention. In yet another embodiment, a composition of the invention comprises at least 10% by weight of one or more crystalline forms of the invention. In certain embodiments, the composition is a pharmaceutical composition comprising a crystalline form of the invention and a pharmaceutically acceptable carrier or excipient.

### Example 1: Preparation of Compound I Crystalline Form A

In a solvent-based screen, the majority of experiments resulted in Compound I Form A, including ambient-temperature slurries in process solvent combinations such as dichloromethane/methyl tert-butyl ether and ethyl acetate/heptane. Crystallization of the amorphous solid also resulted in Form A. Below is a process on multi-kilogram scale to make crystalline Form A of Compound I.

Example 1a: Crystallization of Form A from EtOH: Compound I (crystalline or amorphous, 1 wt.) is charged to the first reactor; Absolute ethanol (200 proof, 55-60 volumes) is charged to the second reactor through a 1 micron PTFE polish filter and heated to 55±5° C. under nitrogen. The pre-heated EtOH from reactor 2 is transferred into reactor 1 under nitrogen. The contents of the first reactor are stirred for ≤20 minutes at 55±5° C. until complete dissolution is obtained (typically within 10-20 minutes). Once fully dissolved, the entire solution is then polished filtered back into the second reactor through a 1 micron PTFE inline filter under nitrogen while maintaining the internal temperature of the solution in the range 30-50° C. (Aim to complete within 30-60 minutes). Under these conditions and dilution, premature nucleation of Compound I was not observed. The internal volume was reduced to 10-15 volumes under reduced pressure at 35-45° C. internal temperature. The vacuum was released, and the slurry placed under nitrogen. A 7 hour time limit is set from the start of dissolution to the completion of condensation of EtOH to minimize racemization at elevated temperature. The product generally will self-nucleate at these reduced volumes, however the solution/mixture can be seeded at this point with ˜1 wt % of Compound I Form A if needed. The batch was cooled to 0-5° C. over ≥2 hours with stirring. The resulting slurry was aged at 0-5° C. for ≥2 hours. The solid is filtered under vacuum and washed with cold (<10° C.) ethanol (absolute, 200 proof, 5 volumes). The cake is de-liquored under vacuum for ≥2 hours, then unloaded into drying trays and transferred to a vacuum oven. The solid was dried under vacuum at 30±5° C. (target 30° C.) and monitored by KF until a water content≤0.5%. The isolated yield is typically 80-90% and the XRPD conforms with Form A. Typical purity is >98% (>99% chiral purity) by HPLC area %.

Example 1b: Crystallization of Form A by dissolution in DCM solution and solvent switch to IPOAc: Crude Compound I (crystalline or amorphous, 1 wt.) dissolved in DCM (˜8 volumes) was concentrated by evaporation at ≤35° C. to ˜5 volumes. IPOAc (5 volumes) was charged to the DCM solution and the solution concentrated to ˜5 volumes at ≤35° C. This operation (addition of 5 volumes of IPOAc followed by concentration to 5 volumes) was repeated 3 more times, affording a slurry of compound I in IPOAc. The slurry was stirred overnight at 23±3° C. The suspension was subsequently cooled to 5±5° C. over 1 hour and the slurry stirred for an additional 3 hours. Solids were collected by filtration and the reactor and cake were washed with chilled IPOAc (3×1 volume). The wet cake was dried under vacuum at 35° C. to afford Compound I Form A. Typical yields are 65-75% and typical purity is >98% (>99% chiral purity) by HPLC area %.

Example 1c: Crystallization of Form A by dissolution in Acetone/EtOH and crystallization from EtOH: crude Compound I (crystalline or amorphous, 1 wt.). was suspended in 20 volumes of acetone and heated to 55±5° C. until full dissolution occurred (<30 min). The warm solution was cooled to 30±5° C. and then concentrated to ˜10 volumes. Ethanol (20 volumes) was then added and the solution again concentrated to ˜10 volumes at ≤35° C. In the course of concentration, the product began to crystallize. The ethanol addition/concentration cycle was repeated 2 more times. The suspension obtained was cooled to 5±5° C. over 1 hour and the slurry was stirred for an additional 2 hours. Solids were collected by filtration and the reactor and cake washed with chilled EtOH (2 volumes). The wet cake was dried under vacuum at 35° C. until residual EtOH was ≤5000 ppm to afford Compound I form A. Typical yields are 80-90% and typical purity is >98% (>99% chiral purity) by HPLC area %.

X-ray powder diffraction pattern and XRPD peaks with relative intensities of the crystalline form thus prepared are shown as in FIG. 1 and Table 2.

### Example 2: Preparation of Compound I Crystalline Forms D, F, and G

The preparation of Form G, including scale-up, following the pathway identified from the screen, includes three steps: generating Form D by slurrying Form A in anhydrous methanol or aqueous methanol at low water activity; generating Form F by air drying Form D; and finally, generating Form G by slurrying Form F in ethyl acetate.

Step 1, to Form D: To ensure sufficient solids of Form D for the next step, four methanol slurries were prepared using Form A solids. Among them, solids were isolated from the 500-mg scale slurry in anhydrous methanol. The solids were isolated after 7 days stirring by vacuum filtration, which yielded a mixture of Forms F and D. Form F is the predominant form in the mixture.

Step 2, to Form F: The Form F/D mixture prepared in the first step was air dried to approximately constant weight. A single phase of Form F was obtained by XRPD.

Step 3, to Form G: 181 mg of Form F from above step 2 was slurried in ethyl acetate at ambient temperature for 7 days. A portion of the solids was isolated and analyzed by XRPD as wet. Based on XRPD, the solids are consistent with Form G, indicating a complete conversion from Form F to Form G in the sample. The remaining solids from the slurry were isolated by vacuum filtration, followed by vacuum drying at ambient temperature to approximately constant weight. The final solids are also consistent with a single phase of Form G, as shown in FIG. 7 (XRPD pattern) and Table 5 (XRPD peak list).

In summary, this preliminary polymorph screen of Compound I produced the preferred form (Form A), and other crystalline Forms D, F, and G, in addition to amorphous material. The screen also identified three (3) other crystalline materials: including potential toluene solvate Material B, Material C, and Material E.

Form A is an anhydrous and unsolvated form of Compound I. It is non-hygroscopic and appears to be stable below 100% RH.

Form D is a methanolate, which converts to Form F upon air drying. Slurrying Form F in ethyl acetate yields Form G, which is anhydrous/unsolvated but may be a racemized material.

Materials C and E were isolated in mixtures with Form A but appear to consist of distinct crystalline phases based on Pawley refinement. Material C was isolated from ethanol solutions and could be an ethanol solvate, which appears to desolvate readily to Form A. Material E was obtained by slurrying in aqueous tetrahydrofuran.

### ABBREVIATIONS

DSC: Differential Scanning calorimetry
DVS: Dynamic Vapor Sorption
FT-IR: Fourier-Transform Infrared
PLM: polarized light microscopy
TGA: thermogravimetric analysis
RH: Relative Humidity
PEG: Polyethylene glycol
XRPD: X-ray powder diffraction
¹H-NMR: proton nuclear magnetic resonance
MEK: methyl ethyl ketone
EtOH: ethanol
H₂O: water
DMSO: dimethyl sulfoxide
EtOAc: ethyl acetate
HCl: hydrogen chloride
ACN: acetonitrile
DCM: dichloromethane
Dioxane: 1,4-dioxane; Ether diethyl ether
Heptane: n-heptane
Hexane: n-hexane
IPA: isopropanol
IPOAc: isopropyl acetate
MeOH: methanol
MIBK: methyl isobutyl ketone
THF: tetrahydrofuran

While this invention has been particularly shown and described with references to preferred embodiments thereof, it will be understood by those skilled in the art that various changes in form and details may be made therein without departing from the scope of the invention encompassed by the appended claims.

1. A crystalline Form A of Compound I, wherein said crystalline form is characterized by an XRPD pattern having characteristic peaks at values of two theta (° 2θ) of 7.04±0.20, 9.22±0.20, 9.64±0.20, 12.89±0.20, 13.61±0.20, 14.28±0.20, 16.33±0.20, 16.78±0.20, 17.10±0.20, 17.46±0.20, 18.03±0.20, 18.52±0.20, 18.85±0.20, 19.37±0.20, 19.68±0.20, 20.16±0.20, 20.62±0.20, 21.21±0.20, 21.68±0.20, 22.11±0.20, 22.66±0.20, 22.98±0.20, 23.15±0.20, 24.71±0.20, 25.38±0.20, 25.97±0.20, 26.36±0.20, and 28.83±0.20.

2. The crystalline form of claim 1, wherein said crystalline form is characterized by an XRPD pattern with characteristic peaks at values of two theta (° 2θ) of 7.04, 9.22, 9.64, 12.89, 13.61, 14.28, 16.33, 16.78, 17.10, 17.46, 18.03, 18.52, 18.85, 19.37, 19.68, 20.16, 20.62, 21.21, 21.68, 22.11, 22.66, 22.98, 23.15, 24.71, 25.38, 25.97, 26.36, and 28.83.

3. The crystalline form of claim 1, wherein said crystalline form is characterized by an XRPD pattern having characteristic peaks at values of two theta (° 2θ) of 7.04±0.20, 9.22±0.20, 9.64±0.20, 11.07±0.20, 11.43±0.20, 12.39±0.20, 12.89±0.20, 13.61±0.20, 14.28±0.20, 16.33±0.20, 16.78±0.20, 17.10±0.20, 17.46±0.20, 18.03±0.20, 18.52±0.20, 18.85±0.20, 19.37±0.20, 19.68±0.20, 19.89±0.20, 20.16±0.20, 20.62±0.20, 21.21±0.20, 21.68±0.20, 22.11±0.20, 22.66±0.20, 22.98±0.20, 23.15±0.20, 24.71±0.20, 24.94±0.20, 25.38±0.20, 25.97±0.20, 26.36±0.20, 26.80±0.20, 27.45±0.20, 27.92±0.20, 28.12±0.20, 28.27±0.20, 28.54±0.20, 28.83±0.20, and 29.40±0.20.

4. The crystalline form of claim 1, wherein said crystalline form is characterized by an XRPD pattern with characteristic peaks at values of two theta (° 2θ) of of 7.0, 9.2, 9.6, 11.1, 11.4, 12.4, 12.9, 13.6, 14.3, 16.3, 16.8, 17.1, 17.5, 18.0, 18.5, 18.9, 19.4, 19.7, 19.9, 20.2, 20.6, 21.2, 21.7, 22.1, 22.7, 23.0, 23.2, 24.7, 25.0, 25.4, 26.0, 26.4, 26.8, 27.5, 27.9, 28.1, 28.3, 28.5, 28.8, and 29.4.

5. A crystalline Form A of Compound I, wherein said crystalline form is characterized by the XRPD pattern set forth in FIG. 1.

6. A composition comprising the crystalline form according to claim 1.

7. A pharmaceutical composition comprising the crystalline form of claim 1 and a pharmaceutically acceptable carrier or excipient.

8. A process for preparing a solution of Compound I, comprising dissolving the crystalline form according to claim 1 in a solvent.
